(19) United States
(12) Patent Application Publication
(10) Pub. No.: US 2026/0215912 A1
(43) Pub. Date: Jul. 30, 2026

(54) PROSTHETIC HEART VALVE

(71) Applicant: Medtronic, Inc., Minneapolis, MN (US)

(72) Inventors: Victoria T. TIEN, Lakeville, MN (US); Karl L. OLNEY, Irvine, CA (US); Alkindi KIBRIA, Irvine, CA (US)

(21) Appl. No.: 19/146,027

(22) PCT Filed: Jan. 17, 2024
(86) PCT No.: PCT/US2024/011847

§ 371 Date: Jul. 7, 2025

Related U.S. Application Data
(60) Provisional application No. 63/441,035, filed on Jan. 25, 2023.

(51) Int. Cl. A61F 2/24

(52) U.S. Cl. CPC A61F 2/2418; A61F 2/243; A61F 2220/0075; A61F 2230/0006; A61F 2250/001; A61F 2250/0036; A61F 2250/0037

(57) ABSTRACT

A prosthetic heart valve includes an annular frame extending along a valve axis. The annular frame includes a plurality of frame members and is adjustable between a radially-collapsed position and a radially-expanded position. A first frame member includes a channel. The first frame member includes a first thickness at the channel, and a second thickness adjacent to the channel. The first thickness is different than the second thickness. A second frame member is pivotably attached to the first frame member. A valve sub-assembly is positioned within an inner passage of the frame. The valve sub-assembly includes a skirt attached to the plurality of frame members, and a plurality of leaflets attached to the skirt. An attachment member is attached to one of the skirt or the plurality of leaflets and extends through the channel such that the attachment member attaches the valve sub-assembly to the frame.

### CROSS-REFERENCE TO RELATED APPLICATIONS

This application claims the benefit of U.S. Provisional Application No. 63/441,035, filed Jan. 25, 2023, the entire contents of which are incorporated herein by reference.

### FIELD

The present disclosure relates generally to a prosthetic heart valve and, more particularly, to a prosthetic heart valve comprising one or more attachment members that attach a valve sub-assembly to a frame.

### BACKGROUND

It is known to provide a prosthetic heart valve for implantation within a target site of the vasculature of a patient. The heart valve prosthesis can be moved from a radially-collapsed position to a radially-expanded position. The heart valve prosthesis can comprise a frame and a valve sub-assembly, such as, for example, a skirt and a plurality of leaflets. Attachment of the valve sub-assembly to the frame can be difficult and time-consuming.

### SUMMARY

The following presents a simplified summary of the disclosure to provide a basic understanding of some aspects described in the detailed description.

In aspects, a heart valve prosthesis comprises an annular frame extending along a valve axis between a first end of the heart valve prosthesis and a second end of the heart valve prosthesis. The annular frame comprises a plurality of frame members and is configured to be adjustable between a radially-collapsed position and a radially-expanded position. The plurality of frame members comprise a first frame member comprising a channel defined along a first side of the first frame member. The first frame member comprises a first thickness, measured along a thickness axis perpendicular to the valve axis, at the channel, and a second thickness adjacent to the channel. The first thickness is different than the second thickness. A second frame member is pivotably attached to the first frame member. The first side of the first frame member faces the second frame member. The heart valve prosthesis comprises a valve sub-assembly positioned within an inner passage of the annular frame. The valve sub-assembly comprises a skirt circumscribing the inner passage and attached to the plurality of frame members, and a plurality of leaflets attached to the skirt. The heart valve prosthesis comprises an attachment member attached to one of the skirt or the plurality of leaflets and extending through the channel such that the attachment member attaches the valve sub-assembly to the frame.

In aspects, the first side of the first frame member comprises a first frame surface extending along a first plane and a second frame surface extending along a second plane. The channel is positioned between the first frame surface and the second frame surface such that the attachment member is located on a side of the first plane and the second plane and is spaced a distance apart from the first plane and the second plane.

In aspects, the first plane is co-planar with the second plane.

In aspects, the channel is bounded by a first channel surface, a second channel surface that is parallel to the first channel surface, and a third channel surface. The first channel surface is attached to the first frame surface. The second channel surface is spaced apart from the first channel surface and is attached to the second frame surface. The third channel surface is substantially parallel to one or more of the first plane or the second plane.

In aspects, the first frame member further comprises a second channel defined along a second side that is opposite the first side. The attachment member extends through the channel and the second channel.

In aspects, the second channel is axially offset from the channel in a lengthwise-direction of the first frame member.

In aspects, the first frame member comprises a hook defining the channel.

In aspects, a prosthetic heart valve prosthesis comprises an annular frame extending along a valve axis between a first end of the heart valve prosthesis and a second end of the heart valve prosthesis. The annular frame comprises a plurality of frame members and is configured to be adjustable between a radially-collapsed position and a radially-expanded position. A first frame member of the plurality of frame members comprises a first opening extending through the first frame member from a first side to a second side. A valve sub-assembly is positioned within an inner passage of the annular frame. The valve sub-assembly comprises a skirt circumscribing the inner passage and attached to the plurality of frame members and a plurality of leaflets attached to the skirt. An attachment member is attached to one of the skirt or the plurality of leaflets and extends through the first opening such that the attachment member attaches the valve sub-assembly to the frame.

In aspects, a radial axis intersects the valve axis and the first frame member. The first opening extends along a transverse axis that is substantially perpendicular to the radial axis.

In aspects, the plurality of frame members comprises a second frame member pivotably attached to the first frame member. The second frame member comprises a second opening extending through the second frame member and along the transverse axis. The attachment member extends through the first opening and the second opening.

In aspects, the transverse axis is oriented in a circumferential direction about the valve axis.

In aspects, a radial axis intersects the valve axis and the first frame member. The first opening extends along the radial axis.

In aspects, the first frame member comprises a second opening extending through the first frame member from the first side to the second side. The second opening is spaced a distance apart from the first opening and extends substantially parallel to the first opening. The attachment member extends through the first opening and the second opening.

In aspects, the first frame member is pivotable at a first node and a second node. The first opening and the second opening are positioned between the first node and the second node.

In aspects, the first frame member is pivotable at a first node and a second node. The first opening is equidistant from the first node and the second node.

In aspects, the attachment member comprises an enlarged portion comprising a member cross-sectional size that is greater than an opening cross-sectional size of the first opening. The enlarged portion is positioned on an outer radial side of the frame member.

In aspects, a prosthetic heart valve prosthesis comprises an annular frame extending along a valve axis between a first end of the heart valve prosthesis and a second end of the heart valve prosthesis. The annular frame comprises a plurality of frame members and is configured to be adjustable between a radially-collapsed position and a radially-expanded position. An expansion apparatus is attached to an interior surface of the frame and extends along an expansion axis parallel to the valve axis. The expansion apparatus is configured to move the frame from the radially-collapsed position to the radially-expanded position. The expansion apparatus comprises an expansion opening extending through the expansion apparatus from a first side to a second side. A valve sub-assembly is positioned within an inner passage of the annular frame. The valve sub-assembly comprises a skirt circumscribing the inner passage and attached to the plurality of frame members and a plurality of leaflets attached to the skirt. An attachment member is attached to one of the skirt or the plurality of leaflets and extends through the expansion opening such that the attachment member attaches the valve sub-assembly to the expansion apparatus.

In aspects, the expansion apparatus comprises a second expansion opening spaced apart from the expansion opening. The attachment member extends through the expansion opening and the second expansion opening.

In aspects, the attachment member extends circumferentially around the expansion apparatus.

In aspects, the expansion apparatus comprises a hook defining the expansion opening.

Additional features and advantages of the aspects disclosed herein will be set forth in the detailed description that follows, and in part will be clear to those skilled in the art from that description or recognized by practicing the aspects described herein, including the detailed description which follows, the claims, as well as the appended drawings. It is to be understood that both the foregoing general description and the following detailed description present aspects intended to provide an overview or framework for understanding the nature and character of the aspects disclosed herein. The accompanying drawings are included to provide further understanding and are incorporated into and constitute a part of this specification. The drawings illustrate various aspects of the disclosure, and together with the description explain the principles and operations thereof.

### BRIEF DESCRIPTION OF THE DRAWINGS

These and other features, aspects and advantages are better understood when the following detailed description is read with reference to the accompanying drawings, in which:

FIG. 1 schematically illustrates example aspects of a heart valve prosthesis in a radially-expanded position in accordance with aspects of the disclosure;

FIG. 2 illustrates a perspective view of a valve sub-assembly being attached to a frame of the heart valve prosthesis of FIG. 1 in accordance with aspects of the disclosure;

FIG. 3 illustrates a close-up perspective view of the frame at focus area 3 of FIG. 1 in accordance with aspects of the disclosure;

FIG. 4 illustrates a side view of the frame as viewed along lines 4-4 of FIG. 3 in accordance with aspects of the disclosure;

FIG. 5 illustrates a side view of additional aspects of the frame similar to FIG. 4 in accordance with aspects of the disclosure;

FIG. 6 illustrates a close-up perspective view of the frame similar to FIG. 3 in accordance with aspects of the disclosure;

FIG. 7 illustrates a side view of the frame as viewed along lines 7-7 of FIG. 6 in accordance with aspects of the disclosure;

FIG. 8 illustrates a side view of additional aspects of the frame similar to FIG. 4 in accordance with aspects of the disclosure;

FIG. 9 illustrates a side view of additional aspects of the frame similar to FIG. 8 in accordance with aspects of the disclosure;

FIG. 10 illustrates a close-up side view of the frame comprising one or more hooks in accordance with aspects of the disclosure;

FIG. 11 illustrates a close-up perspective view of the frame at focus area 11 of FIG. 1 in accordance with aspects of the disclosure;

FIG. 12 illustrates a cross-sectional view of an expansion apparatus along lines 12-12 of FIG. 11 in accordance with aspects of the disclosure; and

FIG. 13 schematically illustrates example aspects of a heart valve prosthesis in a radially-collapsed position in accordance with aspects of the disclosure.

### DETAILED DESCRIPTION

Aspects will now be described more fully hereinafter with reference to the accompanying drawings in which example aspects are shown. Whenever possible, the same reference numerals are used throughout the drawings to refer to the same or like parts. However, this disclosure may be embodied in many different forms and should not be construed as limited to the aspects set forth herein.

As used herein, the term “about” means that amounts, sizes, formulations, parameters, and other quantities and characteristics are not, and need not be, exact, but may be approximate and/or larger or smaller, as desired, reflecting tolerances, conversion factors, rounding off, measurement error and the like, and other factors known to those of skill in the art.

Ranges can be expressed herein as from “about” one value, and/or to “about” another value. When such a range is expressed, aspects include from the one value to the other value. Similarly, when values are expressed as approximations by use of the antecedent “about,” it will be understood that the value forms another aspect. It will be further understood that the endpoints of each of the ranges are significant both in relation to the other endpoint, and independently of the other endpoint.

Directional terms as used herein—for example up, down, right, left, front, back, top, bottom, upper, lower, etc.—are made only with reference to the figures as drawn and are not intended to imply absolute orientation.

Unless otherwise expressly stated, it is in no way intended that any methods set forth herein be construed as requiring that its steps be performed in a specific order, nor that with any apparatus, specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred in any respect. This holds for any possible non-express basis for interpretation, including matters of logic relative to arrangement of steps, operational flow, order of components, or orientation of components; plain meaning derived from grammatical organization or punctuation, and; the number or type of aspects described in the specification.

As used herein, the singular forms “a,” “an,” and “the” include plural references unless the context clearly dictates otherwise. Thus, for example, reference to “a” component includes aspects having two or more such components, unless the context clearly indicates otherwise.

The word “exemplary,” “example,” or various forms thereof are used herein to mean serving as an example, instance, or illustration. Any aspect or design described herein as “exemplary” or as an “example” should not be construed as preferred or advantageous over other aspects or designs. Furthermore, examples are provided solely for purposes of clarity and understanding and are not meant to limit or restrict the disclosed subject matter or relevant portions of this disclosure in any manner. It can be appreciated that a myriad of additional or alternate examples of varying scope could have been presented but have been omitted for purposes of brevity.

As used herein, the terms “comprising,” “including,” and variations thereof, shall be construed as synonymous and open-ended, unless otherwise indicated. A list of elements following the transitional phrases comprising or including is a non-exclusive list, such that elements in addition to those specifically recited in the list may also be present.

The terms “substantial,” “substantially,” and variations thereof as used herein are intended to represent that a described feature is equal or approximately equal to a value or description. For example, a “substantially planar” surface is intended to denote a surface that is planar or approximately planar. Moreover, “substantially” is intended to denote that two values are equal or approximately equal. The term “substantially” may denote values within about 10% of each other, for example, within about 5% of each other, or within about 2% of each other.

Modifications may be made to the instant disclosure without departing from the scope or spirit of the claimed subject matter. Unless specified otherwise, “first,” “second,” or the like are not intended to imply a temporal aspect, a spatial aspect, an ordering, etc. Rather, such terms are merely used as identifiers, names, etc. for features, elements, items, etc. For example, a first end and a second end generally correspond to end A and end B or two different ends.

Unless otherwise indicated, the terms “distal” and “proximal” are used in the following description with respect to a position or direction relative to the treating clinician. “Distal” and “distally” are positions distant from or in a direction away from the clinician, and “proximal” and “proximally” are positions near or in a direction toward the clinician. In addition, the term “self-expanding” may be used in the following description with reference to one or more valve or stent structures of the prostheses hereof and is intended to convey that the structures are shaped or formed from a material that can be provided with a mechanical memory to return the structure from a compressed or constricted delivery configuration to an expanded deployed configuration or vice versa. Non-exhaustive exemplary self-expanding materials include stainless steel, a pseudo-elastic metal such as a nickel titanium alloy or nitinol, various polymers, or a so-called super alloy, which may have a base metal of nickel, cobalt, chromium, or other metal. Mechanical memory may be imparted to a wire or stent structure by thermal treatment to achieve a spring temper in stainless steel, for example, or to set a shape memory in a susceptible metal alloy, such as nitinol. Various polymers that can be made to have shape memory characteristics may also be suitable for use in aspects hereof to include polymers such as polynorborene, trans-polyisoprene, styrene-butadiene, and polyurethane. As well poly L-D lactic copolymer, oligo caprylactone copolymer and poly cyclo-octine can be used separately or in conjunction with other shape memory polymers.

Diseases associated with heart valves, such as those caused by damage or a defect, can include stenosis and valvular insufficiency or regurgitation. For example, valvular stenosis causes the valve to become narrowed and hardened which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses generally include a frame or stent and a prosthetic valve mounted within the frame. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

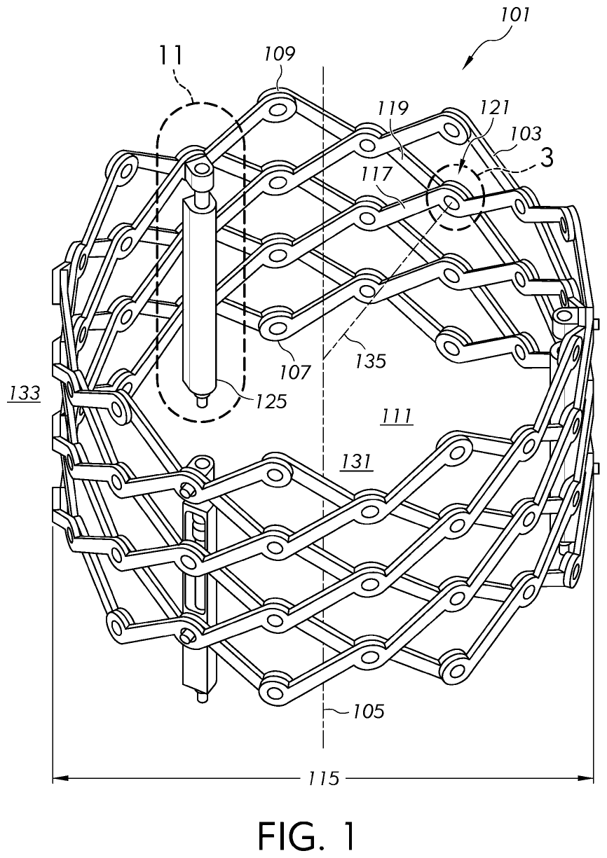

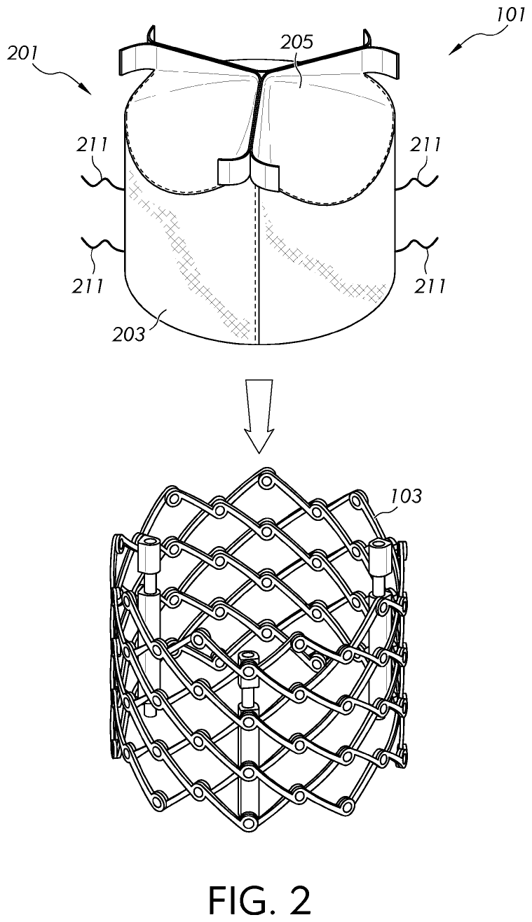

FIG. 1 illustrates a prosthetic annular heart valve prosthesis 101 (hereinafter “valve” or “heart valve” or “valve prosthesis”) that can be delivered to a desired location within a body of a patient, for example, a patient's heart. The valve prosthesis 101 can be moved between a radially-collapsed position and a radially-expanded position, with the valve prosthesis 101 illustrated in the radially-expanded position in FIG. 1. The valve prosthesis 101 can move through a vasculature of the patient while in the radially-collapsed position, and, upon reaching the desired location, can move to the radially-expanded position. The valve prosthesis 101 may comprise one or more structures associated with a heart valve prosthesis, for example, a frame 103, a valve sub-assembly comprising valve leaflets and a skirt (e.g., illustrated in FIG. 2) attached to the frame 103, commissures, etc. For the purposes of illustration and to more clearly depict portions of the frame 103, the valve prosthesis 101 is illustrated without the valve leaflets and the skirt attached to the frame 103, commissures, etc. in FIG. 1. However, in operation, the valve prosthesis 101 comprises the valve leaflets attached to the frame 103, the commissures, etc. In aspects, the valve prosthesis 101 can extend along a valve axis 105 between a first end 107 of the valve prosthesis 101 and a second end 109 of the valve prosthesis 101. The valve prosthesis 101 can comprise an inner passage 111 extending along the valve axis 105 between the first end 107 and the second end 109. The inner passage 111 may be circumferentially bounded by the frame 103.

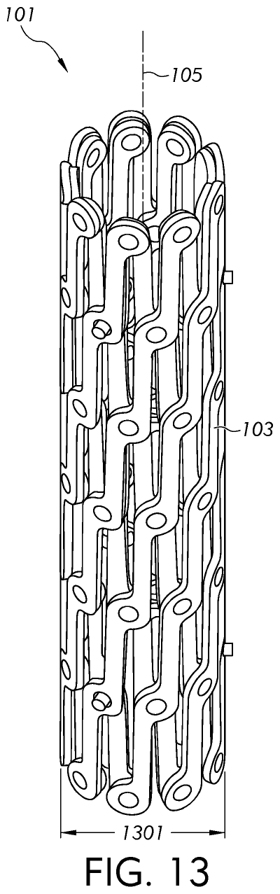

The frame 103 can expand from a radially-collapsed position (e.g., illustrated in FIG. 13) to a radially-expanded position (e.g., illustrated in FIG. 1). In aspects, in the radially-expanded position, the frame 103 can comprise a first diameter 115, and in the radially-collapsed position of FIG. 13, the frame 103 can comprise a second diameter 1301, with the first diameter 115 greater than the second diameter 1301. The frame 103 can comprise a plurality of frame members that may be attached and interconnected. For example, the frame 103 can comprise a first frame member 117, a second frame member 119, etc. The frame members 117, 119 may be pivotably attached at an attachment location or node 121 such that the frame members 117, 119 can be adjustable, for example, by pivoting, between the radially-collapsed position and the radially-expanded position. The frame members 117, 119 can be pivotably attached in several ways, for example, by mechanical fasteners (e.g., rivets, screws, bolts), by snap-fitting without fasteners, etc. and/or may comprise a shape-memory material that can move to a predetermined shape. The frame members 117, 119 (e.g., and the other frame members) may be substantially identical in structure and dimension.

In aspects, the valve prosthesis 101 can comprise an expansion apparatus 125 attached to the frame 103, for example, an interior surface of the frame 103. In aspects, the expansion apparatus 125 can include a hydraulic or pneumatic cylinder, one or more gears, or any number of variations of mechanical or electro-mechanical components that can facilitate movement of the expansion apparatus to expand and/or collapse the valve prosthesis 101. The expansion apparatus 125 can function to mechanically expand the valve prosthesis 101 from the radially-collapsed position to the radially-expanded position. The expansion apparatus 125 can extend substantially parallel to the valve axis 105 between the first end 107 and the second end 109. The expansion apparatus 125 can be attached to one or more of the frame members of the frame 103. The valve prosthesis 101 is not limited to comprising one expansion apparatus 125, but, rather, the valve prosthesis 101 can comprise one or more expansion apparatuses that may be substantially identical to the expansion apparatus 125 described herein. The other expansion apparatuses may be circumferentially spaced apart around the inner passage 111, with the expansion apparatuses attached to an inner radial side or surface of the frame 103. In this way, the expansion apparatus 125 can be positioned at an inner radial side 131 of the frame 103, with the frame 103 defining a boundary between the inner radial side 131 and an outer radial side 133. A radial axis 135 can intersect the valve axis 105 and extend radially from the valve axis 105 and through one or more frame members of the frame 103. For example, starting at the valve axis 105, the radial axis 135 can first pass through the inner radial side 131, followed by passing through the one or more frame members, followed by passing through the outer radial side 133.

FIG. 2 illustrates an exploded view of the valve prosthesis 101 comprising a valve sub-assembly 201 being attached to the frame 103. The valve sub-assembly 201 can comprise a skirt 203 attached to the frame 103, for example, the skirt 203 can be attached to one or more of the plurality of frame members 117, 119, with the skirt 203 circumscribing the inner passage 111 of the annular frame 103. The plurality of leaflets 205 can be attached to one or more of the skirt 203 or the frame 103, for example, the plurality of frame members 117, 119. In aspects, the valve sub-assembly 201 can comprise one or more attachment members 211 attached to one or more of the skirt 203 or the plurality of leaflets 205. The attachment members 211 can attach the valve sub-assembly 201 to the frame 103, for example, when the valve sub-assembly 201 is positioned within the inner passage 111 of the frame 103. It will be appreciated that the valve sub-assembly 201 illustrated in FIG. 2 comprises merely one example of a valve sub-assembly 201 that can be attached to the frame 103 in the manner illustrated and described herein. That is, in aspects, the valve sub-assembly 201 can comprise several different structures and constructions, such as, for example, by being in the form of a tri-leaflet valve (e.g., a tri-leaflet bovine pericardium valve), a bi-leaflet valve, or other suitable valve sub-assemblies. The attachment members 211 can comprise, for example, a flexible loop of material such as a suture or the like. In aspects, the attachment members 211 can comprise one or more of a tissue material, a fabric material, a metal material, a synthetic or natural thread, a polymer, or other elongate material for attaching the valve sub-assembly 201 to the frame 103. Moreover, although illustrated as a single attachment member, it should be understood that, in some aspects, one or more of the attachment members 211 may comprise multiple individual pieces of elongate material or a single piece of elongate material employed more than once as the attachment member. For example, a single loop of one piece of elongate material may define a single attachment member or more than one loop of elongate material may define a single attachment member (e.g., similar to sewing a button on a shirt).

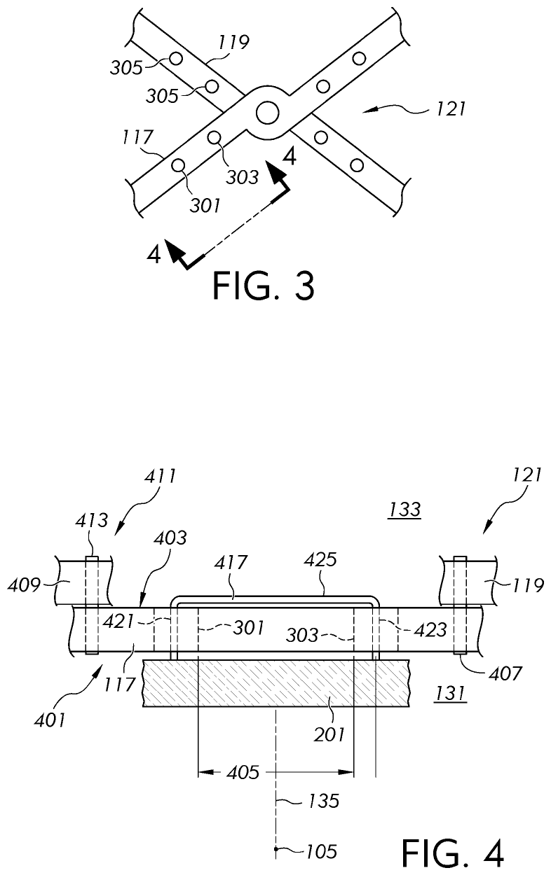

FIG. 3 illustrates a close-up perspective view at focus area 3 of FIG. 1 of the first frame member 117 and the second frame member 119. In aspects, the first frame member 117 and the second frame member 119 can comprise one or more openings, for example, with the first frame member 117 comprising a first opening 301 and a second opening 303 that extend through the first frame member 117, and the second frame member 119 comprising additional openings 305 that extend through the second frame member 119. The openings 301, 303 in the first frame member 117 may be substantially identical to the additional openings 305 in the second frame member 119. Further, in aspects, the other plurality of frame members (e.g., illustrated in FIGS. 1-2) of the frame 103 can comprise openings that may be substantially identical to the openings 301, 303, 305 illustrated and described herein. The first opening 301 and the second opening 303 can be positioned on one side of the node 121 (e.g., the location at which the first frame member 117 is attached to the second frame member 119), with additional openings located on an opposing side of the node 121.

FIG. 4 illustrates a side view of the first frame member 117 as viewed along lines 4-4 of FIG. 3. The first opening 301 and the second opening 303 can extend through the first frame member 117 from a first side 403 to a second side 401. The second side 401 can border or face the inner radial side 131 of the frame 103, and the first side 403 can border or face an opposing direction, for example, the outer radial side 133 of the frame 103 and, thus, may face the second frame member 119. Accordingly, in this way, the radial axis 135 can intersect the valve axis 105 and may intersect the first frame member 117, for example, with the first opening 301 and the second opening 303 extending along the radial axis 135. In aspects, by extending along the radial axis 135, the first opening 301 and the second opening 303 can each extend substantially parallel to the radial axis 135 between the first side 403 and the second side 401. From the perspective illustrated in FIG. 4, the valve axis 105 may extend into and out of the page, while the radial axis 135 can extend vertically through the valve axis 105 and the first frame member 117. The second opening 303 can be spaced a distance 405 apart from the first opening 301, with the first opening 301 and the second opening 303 extending substantially parallel.

The first frame member 117 can be pivotable relative to other frame members at one or more nodes. For example, the first frame member 117 can be attached to and pivotable relative to the second frame member 119 at the first node 121. In aspects, a mechanical fastener 407 (e.g., a screw, a bolt, a rivet, etc.) can attach the first frame member 117 and the second frame member 119 at the first node 121. At another location of the first frame member 117, for example, spaced an axial distance in a length-wise direction of the first frame member 117, the first frame member 117 can be attached to and pivotable relative to another frame member, for example, a third frame member 409 at a second node 411. The first node 121 and the second node 411 may be substantially identical, with the first frame member 117 and the third frame member 409 attached at the second node 411 by a mechanical fastener 413 (e.g., a screw, a bolt, a rivet, etc.). Accordingly, in this way, the first opening 301 and the second opening 303 may be positioned between the first node 121 and the second node 411 of the first frame member 117. That is, an axis extending along the first frame member 117 and intersecting the first node 121 and the second node 411 may intersect the first opening 301 and the second opening 303, with zero nodes positioned between the first opening 301 and the second opening 303. In this way, in aspects, one or more openings may be formed in the first frame member 117 at locations between nodes.

In aspects, an attachment member 417 of the plurality of attachment members 211 (e.g., illustrated in FIG. 2) can extend through the first opening 301 and the second opening 303 such that the attachment member 417 can attach the valve sub-assembly 201 (e.g., the skirt 203 and/or the plurality of leaflets 205) to the frame 103. The attachment member 417 may be attached to the valve sub-assembly 201 at the inner radial side 131. The attachment member 417 can extend radially outwardly and extend through the first opening 301 and the second opening 303. For example, a first member portion 421 of the attachment member 417 can extend through, and be positioned within, the first opening 301. A second member portion 423 of the attachment member 417 can extend through, and be positioned within, the second opening 303. A third member portion 425 can be attached to and extend between the first member portion 421 and the second member portion 423. The third member portion 425 can be located on the first side 403 (e.g., the outer radial side 133) of the first frame member 117, with the third member portion 425 extending adjacent to the first side 403. As illustrated, the third member portion 425 may be separated from the first side 403 by a distance defining a gap, although in other aspects, the third member portion 425 may abut and therefore contact the first side 403 without departing from the scope of the disclosure. Likewise, although the first member portion 421 and the second member portion 423 are shown as being respectively spaced apart from a surface of the inner wall of the first opening 301 and the second opening 303, in aspects, one or both of the first member portion 421 and the second member portion 423 may abut and therefore contact a respective surface of the inner wall of the first opening 301 and/or the second opening 303. In aspects, when the valve prosthesis 101 is in the radially-collapsed position or the radially-expanded position, the frame members may not engage or overlap the third member portion 425 of the attachment member 417. Rather, due to the openings 301, 303 being positioned between the nodes 121, 411, the third member portion 425 may be located a distance apart from the second frame member 119 and/or the third frame member 409. As a result, due to the avoidance of contact between the attachment member 417 and the other frame members 117, 119, 409, damage to the attachment member 417 may be avoided.

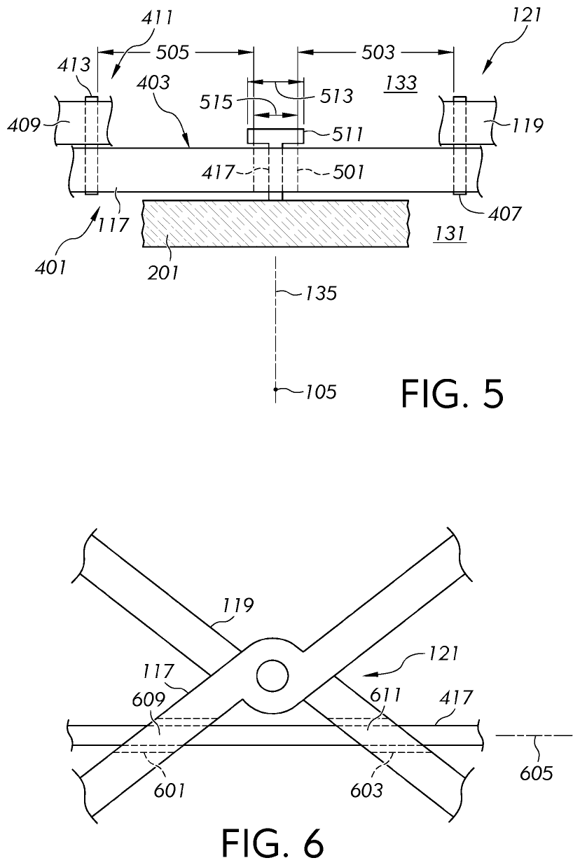

It will be appreciated that for illustrative purposes, the frame members 117, 119, 409, the openings 301, 303, the attachment member 417, etc. illustrated in FIG. 3 are not drawn to scale. Indeed, the distance 405 separating the openings 301, 303, the distance separating the nodes 121, 411, the distance separating the valve axis 105 from the first frame member 117, dimensions of the different structures, etc. may be larger or smaller than as illustrated. As illustrated and described relative to FIG. 5, the first frame member 117 is not limited to comprising the two openings 301, 303, but, rather, can comprise fewer openings (e.g., one opening as illustrated in FIG. 5) or more than two openings. In this way, the other frame members of the frame 103 may be substantially identical in structure and function as the first frame member 117 illustrated and described herein relative to FIGS. 3-10.

FIG. 5 illustrates a side view of the first frame member 117 similar to the aspects illustrated in FIG. 4, but with the first frame member 117 comprising a different number of openings. For example, the first frame member 117 can comprise a first opening 501 that may be equidistant from the first node 121 and the second node 411. By being equidistant, the first opening 501 can be spaced a first distance 503 from the first node 121 (e.g., the fastener 407) and can be spaced a second distance 505 from the second node 411 (e.g., the fastener 413) with zero nodes being located between the first opening 501 and the first node 121 and zero nodes being located between the first opening 501 and the second node 411. In aspects, the first distance 503 may be substantially equal to the second distance 505. In aspects, the attachment member 417 may extend through one opening (e.g., the first opening 501) and may not extend through other openings. To secure the attachment member 417 to the first frame member 117, the attachment member 417 can comprise an enlarged portion 511 comprising a member cross-sectional size 513 that is greater than an opening cross-sectional size 515 of the first opening 501. For example, the enlarged portion 511 can comprise a knot (e.g., a fastening made by tying an end of the attachment member 417) or a fastener attached to the end of the attachment member 417. In aspects, the member cross-sectional size 513 and the opening cross-sectional size 515 can comprise, for example, a maximum cross-sectional size of each of the first opening 501 and the enlarged portion 511. If the first opening 501 and/or the enlarged portion 511 comprises a circular size, then the member cross-sectional size 513 and/or the opening cross-sectional size 515 may comprise a diameter. By being larger in cross-sectional size than the first opening 501, the enlarged portion 511 is limited from passing through the first opening 501, while the remainder of the attachment member 417 (e.g., the portion of the attachment member 417 extending between the valve sub-assembly 201 and the enlarged portion 511) may comprise a cross-sectional size that is smaller than the opening cross-sectional size 515 such that the attachment member 417 can be received within the first opening 501. In this way, the enlarged portion 511 can be positioned on the outer radial side 133 of the first frame member 117, with the attachment member 417 extending from the valve sub-assembly 201 on the inner radial side 131, through the first opening 501, and to the enlarged portion 511 at the outer radial side 133.

FIG. 6 illustrates a close-up perspective view of the first frame member 117 and the second frame member 119 similar to FIG. 3 but with a first opening 601 and a second opening 603 oriented in a different direction than the first opening 301 of FIGS. 3-5. For example, the first frame member 117 can comprise a first opening 601 extending through the first frame member 117, and the second frame member 119 can comprise a second opening 603 extending through the second frame member 119. In aspects, the first opening 601 and the second opening 603 can extend along a transverse axis 605, for example, with the first opening 601 and the second opening 603 oriented substantially parallel to one another and, in aspects, substantially coaxial to one another. To avoid engaging the node 121, the transverse axis 605 can be spaced apart from, and not intersecting, the node 121. In aspects, the transverse axis 605 can be oriented in a circumferential direction about the valve axis 105. For example, the transverse axis 605 can lie at least partially within a circumference of the frame 103, with the frame 103 circling the valve axis 105. The first opening 601 and the second opening 603 can extend along the transverse axis 605 when the valve prosthesis 101 is in the radially-expanded position, and, due to the pivoting of the frame members, may be non-parallel when the valve prosthesis 101 is in the radially-collapsed position. The attachment member 417 can be received within the first opening 601 and the second opening 603, for example, with a first member portion 609 received within the first opening 601 and a second member portion 611 received within the second opening 603.

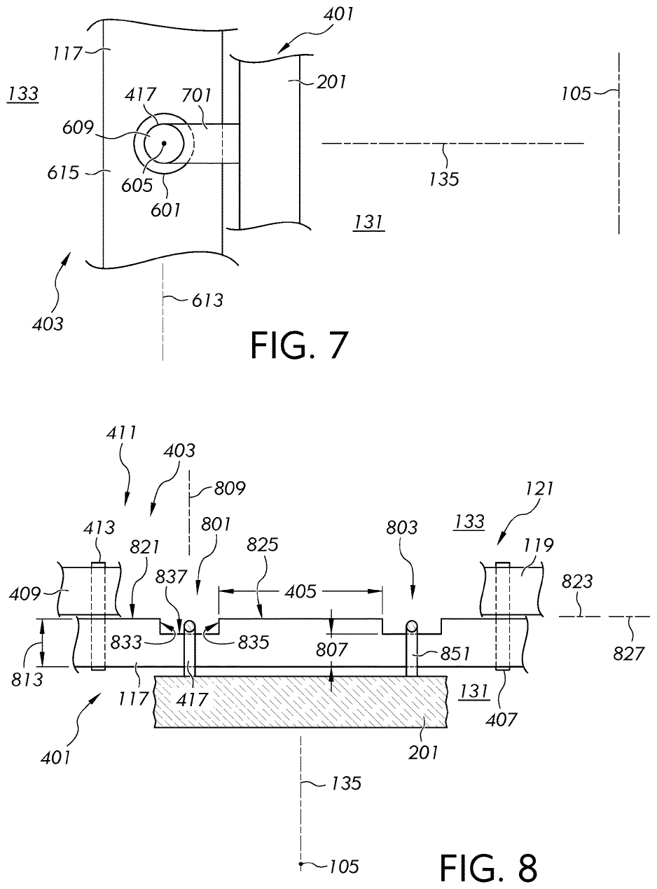

FIG. 7 illustrates a side view of the first frame member 117 as viewed along lines 7-7 of FIG. 6. In aspects, the radial axis 135 can intersect the valve axis 105 and the first frame member 117, with the radial axis 135 intersecting the transverse axis 605. As illustrated in FIG. 7, the transverse axis 605 can extend into and out of the page. In aspects, the transverse axis 605 may be substantially perpendicular to the radial axis 135, such that the transverse axis 605 is not parallel to the valve axis 105 and not parallel to the radial axis 135. The attachment member 417 can comprise a third member portion 701 that extends between, and is attached to, the first member portion 609 at one end and the valve sub-assembly 201 at an opposing end. In this way, the attachment member 417 may not be positioned at, or extend through, the outer radial side 133 of the frame 103. For example, the attachment member 417 may extend from the inner radial side 131 to the first opening 601 of the first frame member 117. The attachment member 417 may then pass through the openings 601, 603 of the frame members 117, 119 without passing through the outer radial side 133 due to the openings 601, 603 being located within an interior of the frame members 117, 119. Likewise, the transverse axis 605 may not pass through the outer radial side 133.

By avoiding the attachment member 417 being located on the outer radial side 133 of the first frame member 117, the attachment member 417 is limited from contacting other frame members. For example, the other frame members (e.g., 119, 409) may be located radially exterior from the first frame member 117, on the outer radial side 133 of the first frame member 117. As such, when the first frame member 117 pivots, the other frame members (e.g., 119, 409) may pivot in a space that occupies the outer radial side 133 of the first frame member 117. However, due to the attachment member 417 extending circumferentially through the first frame member 117 and the second frame member 119 and, thus, not on the outer radial side 133 of the first frame member 117, the attachment member 417 does not contact the other frame members (e.g., 119, 409) that are located radially exterior from the first frame member 117.

By avoiding this contact, inadvertent damage to the attachment member 417 (e.g., from the contact due to pivoting of the other frame members) may be averted. In aspects, the transverse axis 605 is not limited to extending in the circumferential direction about the valve axis 105. Rather, in aspects, the openings 601, 603 can be oriented such that the transverse axis 605 lies in a plane 613 that is substantially perpendicular to the radial axis 135, with the openings 601, 603 bounded by a frame portion 615 on an outer radial side of the openings 601, 603. In this way, the frame portion 615 can limit the attachment member 417 from inadvertently exiting the openings 601, 603 and passing into the outer radial side 133. With the transverse axis 605 lying in the plane 613, the openings 601, 603 can be oriented substantially parallel to the valve axis 105 (e.g., up-and-down in FIG. 7), or at other angles that are perpendicular to the radial axis 135.

FIG. 8 illustrates a side view of the first frame member 117 similar to the aspects illustrated in FIGS. 4-5, but with the first frame member 117 comprising channels instead of openings. For example, as illustrated in FIGS. 3-7, the frame members 117, 119, 409 are illustrated and described as comprising openings 301, 303, 501, 601, 603. The openings 301, 303, 501, 601, 603 can extend through the frame members 117, 119, 409 while being bounded on all sides (e.g., relative to a plane that is perpendicular to an axis along which the openings extend) by a wall of the frame member. In aspects, the openings 301, 303, 501, 601, 603 can comprise circular shapes, quadrilateral shapes (e.g., square, rectangle), or other shapes. In contrast, the first frame member 117 of FIG. 8 can comprise a first channel 801 and a second channel 803, with the channels 801, 803 bounded on some, but not all, sides.

The first frame member 117 (e.g., and the second frame member 119 and the third frame member 409) illustrated in FIG. 8 may be substantially identical in structure to the frame members 117, 119, 409 illustrated and described in FIGS. 3-7, but for the difference between the openings and the channels. For example, the first frame member 117 can comprise the first channel 801 defined along a side (e.g., the first side 403 and/or the second side 401) of the first frame member 117. The first frame member 117 can comprise a first thickness 807, measured along a thickness axis 809 perpendicular to the valve axis 105 and parallel to the radial axis 135, at the first channel 801. The first frame member 117 can comprise a second thickness 813 adjacent to the first channel 801, with the second thickness 813 measured in a direction parallel to the first thickness 807. In aspects, the first thickness 807 is different than the second thickness 813, for example, with the first thickness 807 less than the second thickness 813. In aspects, the first thickness 807 can be measured to surface 837 within the first channel 801 and the second thickness 813 can be measured to surface 821 adjacent to and outside of the first channel 801.

The first side 403 of the first frame member 117 can comprise a first frame surface 821 extending along a first plane 823 and a second frame surface 825 extending along a second plane 827, with the first plane 823 substantially co-planar with the second plane 827. The first channel 801 can be positioned between the first frame surface 821 and the second frame surface 825. In aspects, the first channel 801 can be bordered or defined by a first channel surface 833, a second channel surface 835, and a third channel surface 837. The second channel surface 835 can be parallel to and spaced apart from the first channel surface 833. The first channel surface 833 can be attached to the first frame surface 821, with the first channel surface 833 and the first frame surface 821 forming an angle. The angle may be within a range from about 45 degrees to about 135 degrees, or between about 70 degrees to about 110 degrees, or about 90 degrees. The second channel surface 835 can be attached to the second frame surface 825, with the second channel surface 835 and the second frame surface 825 forming an angle. The angle may be within a range from about 45 degrees to about 135 degrees, or between about 70 degrees to about 110 degrees, or about 90 degrees. In aspects, the third channel surface 837 can extend between the first channel surface 833 and the second channel surface 835, with the third channel surface 837 extending substantially parallel to one or more of the first plane 823 or the second plane 827. In aspects, the third channel surface 837 may be substantially perpendicular to the first channel surface 833 and the second channel surface 835, with the third channel surface 837 recessed from the first frame surface 821 and the second frame surface 825. Accordingly, in this way, the first channel 801 can form a recess, slot, cavity, or the like in a surface of the first frame member 117, with the first channel 801 facing the outer radial side 133 of the first frame member 117.

In aspects, the attachment member 417 can be attached to the valve sub-assembly 201 and can extend through the first channel 801 such that the attachment member 417 can attach the valve sub-assembly 201 to the frame 103. For example, the attachment member 417 can be received within the first channel 801 with the attachment member 417 located on one side of the first plane 823 and the second plane 827. In this way, the attachment member 417 may be spaced a distance apart from the first plane 823 and the second plane 827 while not intersecting the first plane 823 and the second plane 827. Accordingly, the attachment member 417 may lie within the first channel 801 while not being located on the outer radial side 133 of the first frame member 117 (e.g., on an opposite side of the first plane 823 and the second plane 827 from the first channel 801). In this way, the attachment member 417 is limited from contacting other frame members (e.g., 119, 409) such that, when the frame members 117, 119, 409 pivot (e.g., to or from a collapsed or expanded configuration), inadvertent damage to the attachment member 417 may be avoided.

The second channel 803 may be substantially identical in structure to the first channel 801, but for the second channel 803 being spaced the distance 405 apart from the first channel 801. In aspects, the attachment member 417 can be wound around the first frame member 117 with opposing ends of the attachment member 417 attached to the valve sub-assembly 201, and a central region of the attachment member 417 received within the first channel 801. Likewise, a second attachment member 851 can be wound around the first frame member 117 with opposing ends of the second attachment member 851 attached to the valve sub-assembly 201, and a central region of the second attachment member 851 received within the second channel 803. In this way, one attachment member can be received within one channel. Alternatively, the attachment member 417 can be wound around the first frame member 117 such that a first portion of the attachment member 417 can be received within the first channel 801 and a second portion of the attachment member 417 can be received within the second channel 803. In this way, one attachment member (e.g., 417) can be received within multiple channels (e.g., 801, 803).

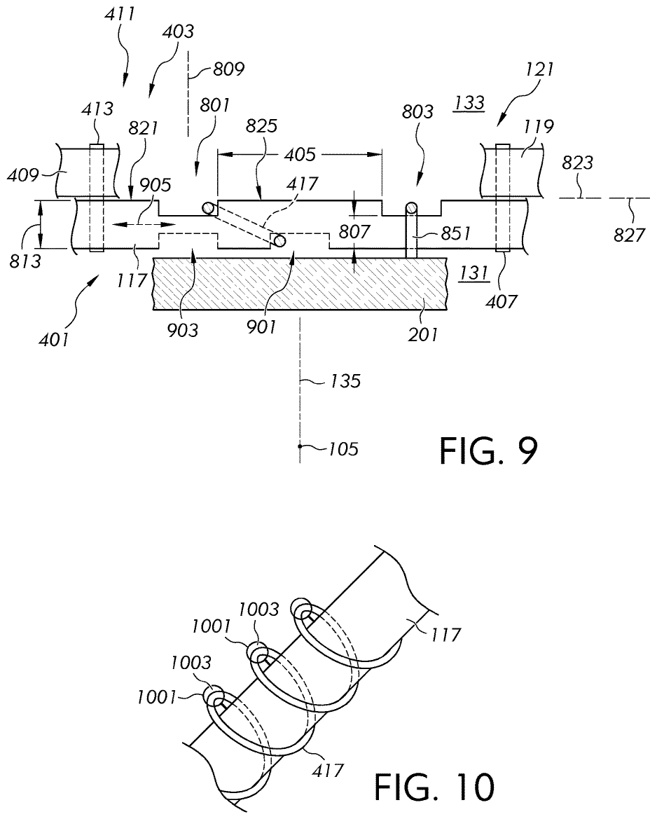

FIG. 9 illustrates a side view of the first frame member 117 similar to the aspects illustrated in FIG. 8, but with additional channels positioned on an opposite side of the first frame member 117 from the first channel 801. For example, with the first channel 801 positioned on the first side 403 of the first frame member 117 (e.g., facing the outer radial side 133), the first frame member 117 can comprise additional channels 901, 903 positioned on the second side 401 facing the inner radial side 131. In aspects, the additional channels 901, 903 may be substantially identical to the first channel 801 in size, shape, and structure. The additional channels 901, 903 are illustrated with dashed lines in FIG. 9 to illustrate possible locations of the additional channels 901, 903 and that, in aspects, the first frame member 117 may or may not comprise one, or both, of the additional channels 901, 903.

In aspects, the first frame member 117 can comprise a second channel 901 defined along the second side 401 that is opposite the first side 403. The second channel 901 can be axially-offset from the first channel 801 in a length-wise direction 905 of the first frame member 117. For example, the length-wise direction 905 can be in a direction along a longest side or length of the first frame member 117 and between the nodes 121, 411. By being axially-offset, the thickness axis 809 that passes through the first channel 801 may not intersect the second channel 901. In this way, the attachment member 417 can be wound around the first frame member 117 and received within (e.g., by extending through) the channels on the first side 403 and the second side 401. For example, on the first side 403, a portion of the attachment member 417 can be received within the first channel 801, and on the second side 401, a different portion of the attachment member 417 can be received within the second channel 901. The attachment member 417 can continue to be wound around the first frame member 117 in a similar manner to attach the valve sub-assembly 201 to the first frame member 117. In addition, or in the alternative, the first frame member 117 is not limited to comprising the second channel 901 that is axially-offset from the first channel 801. Rather, in aspects, the third channel 903 can be aligned with the first channel 801, with the third channel 903 located on an opposite side from the first channel 801. In this way, the thickness axis 809 can intersect the first channel 801 and the third channel 903. As with the second channel 901, the attachment member 417 can be wound around the first frame member 117 and may be received within the first channel 801 and the second channel 901. FIGS. 8-9 illustrate the channels 801, 803, 901, 903 being positioned on one or more sides.

For example, in aspects, one or more channels 801, 803 may be located on one side (e.g., the first side 403) with zero other channels located on the remaining three sides of the first frame member 117. In addition, or in the alternative, in aspects, one or more channels 801, 803 may be located on one side (e.g., the first side 403) and one or more channels 901, 903 may be located on an opposite side (e.g., the second side 401) with zero other channels on the other sides that extend between the first side 403 and the second side 401. In addition, or in the alternative, one or both of the sides that extend between the first side 403 and the second side 401 can comprise channels, which may be substantially identical to the 801, 803, 901, 903 described herein. In this way, the channels 801, 803, 901, 903 are not limited to being positioned on the sides illustrated in FIGS. 8-9, but could be on three sides, or all four sides.

FIG. 10 illustrates additional aspects of the first frame member 117, wherein the first frame member 117 can comprise one or more hooks 1001 attached to a side (e.g., the first side 403, the second side 401, or other sides) of the first frame member 117. The hooks 1001 can comprise a wall that defines channels 1003 within which the attachment member 417 can be received. The hooks 1001 can be spaced apart in a lengthwise direction of the first frame member 117. In this way, the attachment member 417 can be received within the channels 1003 and wound around the first frame member 117. As such, the attachment member 417 can attach the valve sub-assembly 201 to the first frame member 117 by passing through the channels 1003 of the hooks 1001. In aspects, the hooks 1001 can be provided with none, some, or all of the previous aspects (e.g., the openings, the channels) illustrated and described relative to FIGS. 3-9. Likewise, any one or more of the disclosed attachment members and related openings, channels, or hooks may be employed alone or in combination to attach one or more portions of the valve sub-assembly to the frame to provide the heart valve prosthesis 101 contemplated herein.

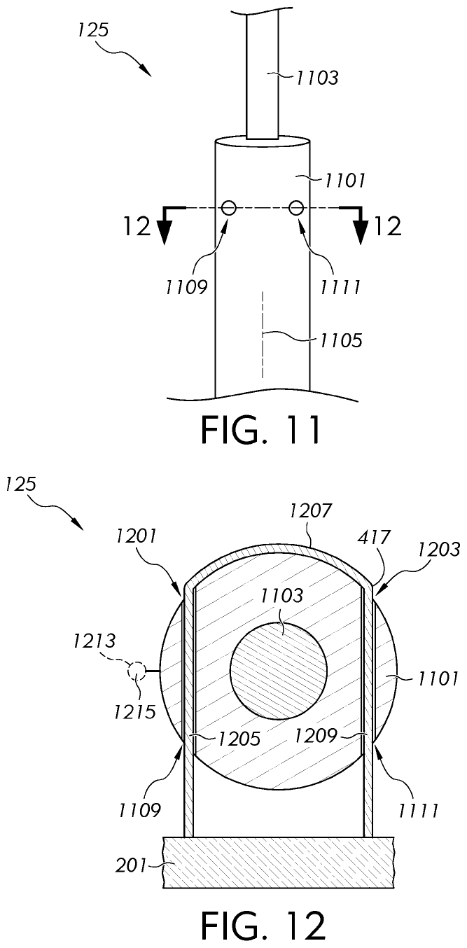

FIG. 11 illustrates a close-up perspective view at focus area 11 of FIG. 1 of the expansion apparatus 125. The expansion apparatus 125 can comprise a first expansion portion 1101 and a second expansion portion 1103. The first expansion portion 1101 may be substantially hollow such that the second expansion portion 1103 can be received within a chamber of the first expansion portion 1101. The second expansion portion 1103 can move, for example, axially, relative to the first expansion portion 1101 along an expansion axis 1105 that is parallel to the valve axis 105. In this way, the second expansion portion 1103 can move along the expansion axis 1105 that is substantially parallel to the valve axis 105. The second expansion portion 1103 can be moved relative to the first expansion portion 1101 in several ways. For example, the second expansion portion 1103 can be threaded and may be received within a corresponding threaded opening of the first expansion portion 1101, such that the second expansion portion 1103 can be rotated to move axially relative to the first expansion portion 1101. In other aspects, the expansion apparatus 125 can comprise a gear within the first expansion portion 1101 such that the second expansion portion 1103 can be gear driven. It will be appreciated that the expansion apparatus 125 can comprise any number of variations that can facilitate movement of one expansion portion relative to the other expansion portion. With the first expansion portion 1101 and the second expansion portion 1103 attached to frame members of the frame 103, movement of the second expansion portion 1103 relative to the first expansion portion 1101 can cause the frame members to move such that the valve prosthesis 101 can mechanically expand from the radially-collapsed position to the radially-expanded position.

In aspects, the expansion apparatus 125 can comprise one or more openings, for example, a first expansion opening 1109 and a second expansion opening 1111. The first expansion opening 1109 and the second expansion opening 1111 can be formed in the first expansion portion 1101, with the second expansion opening 1111 spaced apart, for example, circumferentially apart, from the first expansion opening 1109. FIG. 12 illustrates a cross-sectional view of the expansion apparatus 125 along lines 12-12 of FIG. 11. In aspects, the expansion apparatus 125 can comprise additional openings, for example, a third expansion opening 1201 and a fourth expansion opening 1203. The first expansion opening 1109, the second expansion opening 1111, the third expansion opening 1201, and the fourth expansion opening 1203 may be spaced circumferentially about the expansion axis 1105 and may define a passage from an exterior of the expansion apparatus 125 and through the first expansion portion 1101.

The attachment member 417 can extend through the expansion openings 1109, 1111, 1201, 1203 to attach the valve sub-assembly 201 to the expansion apparatus 125. For example, a first segment 1205 of the attachment member 417 can extend through the first expansion opening 1109 and the third expansion opening 1201, with an end of the first segment 1205 attached to the valve sub-assembly 201. The attachment member 417 can comprise a second segment 1207 that is attached to the first segment 1205 and extends between the third expansion opening 1201 and the fourth expansion opening 1203. While the first segment 1205 is located at least partially within the first expansion portion 1101, the second segment 1207 may be located at an exterior of the first expansion portion 1101. In aspects, the first segment 1205 may extend substantially linearly between the first expansion opening 1109 and the third expansion opening 1201. The second segment 1207 can match the shape of an exterior surface of the first expansion portion 1101 between the third expansion opening 1201 and the fourth expansion opening 1203 such that the second segment 1207 may comprise a non-linear shape, such as a rounded shape. The attachment member 417 can comprise a third segment 1209 that is attached to the second segment 1207 and extends from the second segment 1207 to the valve sub-assembly 201. In aspects, the third segment 1209 can be attached at one end to the second segment 1207 and at an opposing end to the valve sub-assembly 201. The third segment 1209 can extend through the fourth expansion opening 1203 and the second expansion opening 1111 while extending substantially linearly between the fourth expansion opening 1203 and the second expansion opening 1111.

By extending through the expansion openings 1109, 1111, 1201, 1203, the attachment member 417 can extend at least partially circumferentially around the expansion apparatus 125. As such, the valve sub-assembly 201 can be attached to the expansion apparatus 125 with the attachment member 417. Further, due to the expansion openings 1109, 1111, 1201, 1203, the attachment member 417 can extend at least partially within the first expansion portion 1101, such that the first expansion portion 1101 can shield the attachment member 417 from damage. For example, by being shielded, the attachment member 417 can be limited from inadvertently contacting other portions of the valve prosthesis 101, for example, the frame members, that may inadvertently damage the attachment member 417.

In addition, or in the alternative, the expansion apparatus 125 can comprise one or more hooks 1213 that may be substantially identical to the hooks 1001 of FIG. 10. For example, the hook 1213 can comprise a channel 1215 that can receive the attachment member 417. In aspects, the hook 1213 can be attached to an outer radial surface of the first expansion portion 1101. The hook 1213 is illustrated with dashed lines in FIG. 12 to illustrate one possible location of the hook 1213, though, in aspects, the hook 1213 (e.g., or additional hooks) may be positioned at different locations about the outer radial surface of the first expansion portion 1101. To further facilitate attachment of the valve sub-assembly 201 to the expansion apparatus 125, the hook 1213 can receive the attachment member 417 through the channel 1215. In this way, the attachment member 417 is limited from moving (e.g., axially) relative to the expansion apparatus 125, thus limiting the likelihood of unwanted movement of the valve sub-assembly 201 relative to the frame 103.

The frame members illustrated and described herein can provide several benefits. For example, various ways to attach the valve sub-assembly 201 to the first frame member 117 and/or the expansion apparatus 125 can comprise openings, channels, hooks, etc. The attachment member 417 can pass through the openings, channels, hooks, etc. In this way, the attachment member 417 can be at least partially recessed or shielded by portions of the frame member 117 (e.g., relative to FIGS. 3-9), by the hooks (e.g., relative to FIGS. 10 and 12), or by the expansion apparatus 125 (e.g., relative to FIGS. 11-12). In this way, the attachment member 417 can be at least partially protected from damage that may occur from the attachment member 417 contacting other portions of the frame 103. For example, as the frame 103 moves from the radially-contracted position to the radially-expanded position, the frame members can move, for example, by pivoting. Avoiding contact between the frame members and the attachment member 417 can be beneficial due, in part, to the avoidance of damage to the attachment member 417. Accordingly, by at least partially shielding the attachment member 417 from the portions of the frame 103 while the frame 103 moves can reduce inadvertent damage to the attachment member 417.

The openings and/or the channels can be formed in the frame members in several ways. For example, the openings and/or the channels can be formed via a laser-cutting process, a stamping or punching process, etc. In addition, while the openings and/or channels are described relative to the first frame member 117 herein, wherein the openings and/or the channels may be positioned at the outer radial side of the first frame member 117, similar openings and/or channels can be provided on the second frame member 119. For example, the second frame member 119 is located at an outer radial side of the first frame member 117 (e.g., radially exterior from the first frame member 117). In aspects, openings and/or channels can be positioned on the inner radial side of the second frame member 119 (e.g., facing the first frame member 117) to shield attachment members that may be positioned in the openings and/or channels of the second frame member 119. In this way, in aspects, openings and/or channels can be positioned on the side of the frame member (e.g., 117, 119, etc.) that radially faces another frame member. Accordingly, the first frame member 117 can comprise openings and/or channels on an outer radial side of the first frame member 117 facing the second frame member 119. Likewise, the second frame member 119 can comprise openings and/or channels on an inner radial side of the second frame member 119 facing the first frame member 117.

It should be understood that while various aspects have been described in detail relative to certain illustrative and specific examples thereof, the present disclosure should not be considered limited to such, as numerous modifications and combinations of the disclosed features are possible without departing from the scope of the following claims.

What is claimed is:

1. A heart valve prosthesis comprising:
an annular frame extending along a valve axis between a first end of the heart valve prosthesis and a second end of the heart valve prosthesis, the annular frame comprising a plurality of frame members and configured to be adjustable between a radially-collapsed position and a radially-expanded position, the plurality of frame members comprising:
a first frame member comprising a channel defined along a first side of the first frame member, the first frame member comprising a first thickness, measured along a thickness axis perpendicular to the valve axis, at the channel, and a second thickness adjacent to the channel, the first thickness different than the second thickness; and
a second frame member pivotably attached to the first frame member, the first side of the first frame member facing the second frame member;
a valve sub-assembly positioned within an inner passage of the annular frame, the valve sub-assembly comprising a skirt circumscribing the inner passage and attached to the plurality of frame members, and a plurality of leaflets attached to the skirt; and
an attachment member attached to one of the skirt or the plurality of leaflets and extending through the channel such that the attachment member attaches the valve sub-assembly to the frame.

2. The heart valve prosthesis of claim 1, wherein the first side of the first frame member comprises a first frame surface extending along a first plane and a second frame surface extending along a second plane, the channel positioned between the first frame surface and the second frame surface such that the attachment member is located on a side of the first plane and the second plane and spaced a distance apart from the first plane and the second plane.

3. The heart valve prosthesis of claim 2, wherein the first plane is co-planar with the second plane.

4. The heart valve prosthesis of claim 3, wherein the channel is bounded by a first channel surface, a second channel surface that is parallel to the first channel surface, and a third channel surface, the first channel surface attached to the first frame surface, the second channel surface spaced apart from the first channel surface and attached to the second frame surface, and the third channel surface substantially parallel to one or more of the first plane or the second plane.

5. The heart valve prosthesis of claim 2, wherein the first frame member further comprises a second channel defined along a second side that is opposite the first side, the attachment member extending through the channel and the second channel.

6. A heart valve prosthesis comprising:
an annular frame extending along a valve axis between a first end of the heart valve prosthesis and a second end of the heart valve prosthesis, the annular frame comprising a plurality of frame members and configured to be adjustable between a radially-collapsed position and a radially-expanded position, a first frame member of the plurality of frame members comprising a first opening extending through the first frame member from a first side to a second side;
a valve sub-assembly positioned within an inner passage of the annular frame, the valve sub-assembly comprising a skirt circumscribing the inner passage and attached to the plurality of frame members and a plurality of leaflets attached to the skirt; and
an attachment member attached to one of the skirt or the plurality of leaflets and extending through the first opening such that the attachment member attaches the valve sub-assembly to the frame.

7. The heart valve prosthesis of claim 6, wherein a radial axis intersects the valve axis and the first frame member, the first opening extending along a transverse axis that is substantially perpendicular to the radial axis.

8. The heart valve prosthesis of claim 6, wherein a radial axis intersects the valve axis and the first frame member, the first opening extending along the radial axis.

9. The heart valve prosthesis of claim 8, wherein the first frame member comprises a second opening extending through the first frame member from the first side to the second side, the second opening spaced a distance apart from the first opening and extending substantially parallel to the first opening, the attachment member extending through the first opening and the second opening.

10. The heart valve prosthesis of claim 6, wherein the first frame member is pivotable at a first node and a second node, the first opening is equidistant from the first node and the second node.

11. The heart valve prosthesis of claim 6, wherein the attachment member comprises an enlarged portion comprising a member cross-sectional size that is greater than an opening cross-sectional size of the first opening, the enlarged portion positioned on an outer radial side of the frame member.

12. A heart valve prosthesis comprising:
an annular frame extending along a valve axis between a first end of the heart valve prosthesis and a second end of the heart valve prosthesis, the annular frame comprising a plurality of frame members and configured to be adjustable between a radially-collapsed position and a radially-expanded position;
an expansion apparatus attached to an interior surface of the frame and extending along an expansion axis parallel to the valve axis, the expansion apparatus configured to move the frame from the radially-collapsed position to the radially-expanded position, the expansion apparatus comprising an expansion opening extending through the expansion apparatus from a first side to a second side;
a valve sub-assembly positioned within an inner passage of the annular frame, the valve sub-assembly comprising a skirt circumscribing the inner passage and attached to the plurality of frame members and a plurality of leaflets attached to the skirt; and
an attachment member attached to one of the skirt or the plurality of leaflets and extending through the expansion opening such that the attachment member attaches the valve sub-assembly to the expansion apparatus.

13. The heart valve prosthesis of claim 12, wherein the expansion apparatus comprises a second expansion opening spaced apart from the expansion opening, the attachment member extending through the expansion opening and the second expansion opening.

14. The heart valve prosthesis of claim 13, wherein the attachment member extends circumferentially around the expansion apparatus.

15. The heart valve prosthesis of claim 12, wherein the expansion apparatus comprises a hook defining the expansion opening.
